(19) United States
(12) Patent Application Publication
(10) Pub. No.: US 2006/0078889 A1
(43) Pub. Date: Apr. 13, 2006

(54) Array-based methods for producing ribonucleic acids

(76) Inventors: Arindam Bhattacharjee, Loveland, CO (US); Stephanie Fulmer-Smentek, Loveland, CO (US); Diane D. Ilsley, Loveland, CO (US); Eric M. Leproust, Loveland, CO (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/961,991

(22) Filed: Oct. 8, 2004

(51) Int. Cl. C12Q 1/68; G06F 19/00; C12P 19/34

(52) U.S. Cl. 435/6; 435/91.2; 702/20

(57) ABSTRACT

Methods and compositions for generating pluralities of ribonucleic acids are provided. In the subject methods, an array is employed as a template in an in vitro transcription reaction. Also provided are the arrays employed in the subject methods and kits for practicing the subject methods. The ribonucleic acid pluralities produced by the subject methods find use in a variety of different applications, including differential gene expression analysis and gene-silencing applications.

### BACKGROUND OF THE INVENTION

Chemical arrays, such as nucleic acid and protein arrays are finding increasing use in a variety of different applications, and in doing so are making a signicant impact in a variety of different fields, including research, medicine, and the like. In many instances, arrays include regions of usually different composition arranged in a predetermined configuration on a substrate. These regions (sometimes referenced as “features”) are positioned at known respective locations (“addresses”) on the substrate and are therefore “addressable.”

In using such arrays, the arrays are, in many applications, exposed to a sample. Upon sample exposure, the arrays will exhibit an observed binding pattern that is dependent on the sample composition. This observed binding pattern is then detected upon interrogating the array. The observed binding pattern is then employed to determine the presence and/or concentration of one or more polynucleotide components of the sample. Representative methods for sample preparation, labeling, and hybridizing include those disclosed in U.S. Pat. Nos. 6,201,112; 6,132,997; and 6,235,483; as well as published U.S. patent application 20020192650.

Arrays can be fabricated by depositing previously obtained biopolymers onto a substrate, or by in situ synthesis methods. The in situ fabrication methods include those described in U.S. Pat. Nos. 5,449,754 and 6,180,351 as well as published PCT application no. WO 98/41531 and the references cited therein. Further details of fabricating biopolymer arrays are described in U.S. Pat. Nos. 6,242,266; 6,232,072; 6,180,351 and U.S. Pat. No. 6,171,797. Other techniques for fabricating biopolymer arrays include known light directed synthesis techniques.

As the technology of making and using arrays continues to advance, there is a continued interest in the development of new applications for these powerful tools.

### SUMMARY OF THE INVENTION

Methods and compositions for generating pluralities of distinct ribonucleic acids are provided. In the subject methods, a template array is employed in an in vitro transcription reaction to produce a plurality of distinct ribonucleic acids. A feature of the template arrays employed in the subject methods is that they include a plurality of distinct features of surface immobilized nucleic acids made up of a surface proximal RNA polymerase promoter domain and a surface distal variable domain. Also provided are the arrays employed in the subject methods and kits for practicing the subject methods. The ribonucleic acids produced by the subject methods find use in a variety of different applications, including differential gene expression analysis, gene-silencing applications and nucleic acid library generation applications.

In certain aspects of the invention, methods are provided for producing a plurality of ribonucleic acids, where the methods include a first step of contacting: (i) an array of at least two distinct features each including single-stranded nucleic acids immobilized on a surface of a solid support and having a surface proximal RNA polymerase promoter domain and a surface distal variable domain; with (ii) nucleic acids complementary to the RNA polymerase promoter domain of the single-stranded nucleic acids of the features; to produce a template array of overhang duplex nucleic acids, wherein each overhang duplex nucleic acid of the resultant array includes a double-stranded RNA polymerase promoter region and a single-stranded variable region overhang. The resultant template array is then subjected to an in vitro transcription protocol to produce a product plurality of ribonucleic acids of differing sequence. In certain embodiments, the single-stranded surface immobilized nucleic acids of the array further include a linking domain between said promoter and variable domains. In certain embodiments, the single-stranded surface immobilized nucleic acids of the array further may include a spacer between the surface proximal RNA polymerase promoter domain and the substrate surface. In certain embodiments, the immobilized nucleic acids of the features of the array each have the same RNA polymerase promoter domain. In certain embodiments, the RNA polymerase promoter domain is chosen from a T7, T3 and SP6 polymerase promoter domain. In certain embodiments, the method further includes subjecting the template array product to primer extension reaction conditions prior to subjecting the template array to the in vitro transcription reaction conditions. In certain embodiments, the method further includes separating the product mixture from the template array. In certain embodiments, the product plurality is labeled, while in other embodiments it is not labeled. In certain embodiments, the single stranded surface immobilized nucleic acids of the features of the array are described by the formula:
surface-Sₛ-R-L_{I}-V-5′
wherein:
S is a spacer domain;
s is an integer of 0 or 1;
R is said surface proximal RNA polymerase promoter domain;
L is a linking domain;
I is an integer of 0 or 1; and
V is said surface distal variable domain.

In representative embodiments, the subject arrays have a feature density ranging from about 1000 to about 10,000 features/cm², such as from about 2,000 to about 10,000 features/cm², including from about 2,000 to about 5,000 features/cm².

In representative embodiments, the density of single-stranded nucleic acids within a given feature is selected to optimize efficiency of the RNA polymerase. In certain of these representative embodiments, the density of the single-stranded nucleic acids may range from about 10⁻³ to about 1 pmol/mm², such as from about 10⁻² to about 0.1 pmol/mm², including from about 5×10⁻² to about 0.1 pmol/mm².

In certain embodiments, the method further includes employing the product mixture in a differential gene expression analysis application. In certain embodiments, the method further includes employing the product mixture in a gene-silencing application. In certain embodiments, the method further includes employing the product mixture in a nucleic acid library generation application.

Also provided by the invention are arrays that include at least two distinct nucleic acid features each including single-stranded nucleic acids immobilized on a surface of substrate, wherein each of the surface immobilized single-stranded nucleic acids includes a surface proximal RNA polymerase promoter domain and a surface distal variable domain. In certain embodiments, the arrays are further characterized by one or more of the additional features as reviewed above in connection with the description of the subject methods.

Also provided is a template array that includes at least two distinct nucleic acid features each including surface immobilized overhang duplex nucleic acids, wherein each overhang duplex nucleic acid of the array includes a double-stranded RNA polymerase promoter region and a single-stranded variable region overhang. In certain embodiments, the arrays are further characterized by one or more of the additional features as reviewed above in connection with the description of the subject methods.

Also provided are kits for use in producing a mixture of ribonucleic acids, where the kits include: (a) an array that includes at least two distinct nucleic acid features each including single-stranded nucleic acids immobilized on a surface of substrate, wherein each of the surface immobilized single-stranded nucleic acids includes a surface proximal RNA polymerase promoter domain and a surface distal variable domain; and (b) nucleic acids complementary to the RNA polymerase promoter domain. In certain embodiments, the kit further includes a RNA polymerase. In certain embodiments, the kit further includes ribonulcleotides, where in certain embodiments the ribonucleotides are labeled. In certain embodiments, the kit components are further characterized by one or more of the additional features, as reviewed above in connection with the description of the subject methods.

Also provided are methods of detecting the presence of a nucleic acid analyte in a sample, where the methods include: (a) producing from the sample a target composition that includes: (i) labeled deoxyribonucleic acid target molecules labeled with a first label; and (ii) a ribonucleic acid reference labeled with a second label distinguishable from the first label, where the reference is produced according to the method of of the invention; (b) contacting the target composition with a nucleic acid array; and (c) detecting any binding complexes on the surface of the the array to determine the presence of the nucleic acid analyte in said sample. In certain embodiments, the method further includes a data transmission step in which a result from a reading of the array is transmitted from a first location to a second location. In certain embodiments, the second location is a remote location. Also provided are methods of receiving data representing a result of a reading obtained by the method above described methods.

### BRIEF DESCRIPTION OF THE FIGURES

FIG. 1 provides a schematic view of a representative embodiment of the subject methods.

FIG. 2 provides a schematic view of a second representative embodiment of the subject methods.

### DEFINITIONS

A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. As such, this term includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. Specifically, a “biopolymer” includes deoxyribonucleic acid or DNA (including cDNA), ribonucleic acid or RNA and oligonucleotides, regardless of the source.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “mRNA” means messenger RNA.

A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Nucleotide sub-units of deoxyribonucleic acids are deoxyribonucleotides, and nucleotide sub-units of ribonucleic acids are ribonucleotides.

An “oligonucleotide” generally refers to a nucleotide multimer of about 10 to 100 nucleotides in length, while a “polynucleotide” includes a nucleotide multimer having any number of nucleotides.

A chemical “array”, unless a contrary intention appears, includes any one, two or three-dimensional arrangement of addressable regions bearing a particular chemical moiety or moieties (for example, biopolymers such as polynucleotide sequences) associated with that region, where the chemical moiety or moieties are immobilized on the surface in that region. By “immobilized” is meant that the moiety or moities are stably associated with the substrate surface in the region, such that they do not separate from the region under conditions of using the array, e.g., hybridization and washing conditions. As is known in the art, the moiety or moieties may be covalently or non-covalently bound to the surface in the region. For example, each region may extend into a third dimension in the case where the substrate is porous while not having any substantial third dimension measurement (thickness) in the case where the substrate is non-porous. An array may contain more than ten, more than one hundred, more than one thousand more than ten thousand features, or even more than one hundred thousand features, in an area of less than 20 cm² or even less than 10 cm². For example, features may have widths (that is, diameter, for a round spot) in the range of from about 10 μm to about 1.0 cm. In other embodiments each feature may have a width in the range of about 1.0 μm to about 1.0 mm, such as from about 5.0 μm to about 500 μm, and including from about 10 μm to about 200 μm. Non-round features may have area ranges equivalent to that of circular features with the foregoing width (diameter) ranges. A given feature is made up of chemical moieties, e.g., nucleic acids, that bind to (e.g., hybridize to) the same target (e.g., target nucleic acid), such that a given feature corresponds to a particular target. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide. Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. The total number of oligonucleotide molecules per features is extremely important. An array is “addressable” in that it has multiple regions (sometimes referenced as “features” or “spots” of the array) of different moieties (for example, different polynucleotide sequences) such that a region at a particular predetermined location (an “address”) on the array will detect a particular target or class of targets (although a feature may incidentally detect non-targets of that feature). The target for which each feature is specific is, in representative embodiments, known. An array feature is generally homogenous in composition and concentration and the features may be separated by intervening spaces (although arrays without such separation can be fabricated).

In the case of an array, the “target” will be referenced as a moiety in a mobile phase (typically fluid), to be detected by probes (“target probes”) which are bound to the substrate at the various regions. However, either of the “target” or “target probes” may be the one which is to be detected by the other (thus, either one could be an unknown mixture of polynucleotides to be detected by binding with the other). “Addressable sets of probes” and analogous terms refer to the multiple regions of different moieties supported by or intended to be supported by the array surface.

An “array layout” or “array characteristics”, refers to one or more physical, chemical or biological characteristics of the array, such as positioning of some or all the features within the array and on a substrate, one or more feature dimensions, or some indication of an identity or function (for example, chemical or biological) of a moiety at a given location, or how the array should be handled (for example, conditions under which the array is exposed to a sample, or array reading specifications or controls following sample exposure).

“Hybridizing” and “binding”, with respect to polynucleotides, are used interchangeably.

A “plastic” is any synthetic organic polymer of high molecular weight (for example at least 1,000 grams/mole, or even at least 10,000 or 100,000 grams/mole.

“Flexible” with reference to a substrate or substrate web (including a housing or one or more housing component such as a housing base and/or cover), references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C. “Rigid” refers to a substrate (including a housing or one or more housing component such as a housing base and/or cover) which is not flexible, and is constructed such that a segment about 2.5 by 7.5 cm retains its shape and cannot be bent along any direction more than 60 degrees (and often not more than 40, 20, 10, or 5 degrees) without breaking.

When one item is indicated as being “remote” from another, this descriptor indicates that the two items are at least in different buildings, and may be at least one mile, ten miles, or at least one hundred miles apart. When different items are indicated as being “local” to each other they are not remote from one another (for example, they can be in the same building or the same room of a building). “Communicating”, “transmitting” and the like, of information reference conveying data representing information as electrical or optical signals over a suitable communication channel (for example, a private or public network, wired, optical fiber, wireless radio or satellite, or otherwise). Any communication or transmission can be between devices which are local or remote from one another. “Forwarding” an item refers to any means of getting that item from one location to the next, whether by physically transporting that item or using other known methods (where that is possible) and includes, at least in the case of data, physically transporting a medium carrying the data or communicating the data over a communication channel (including electrical, optical, or wireless). “Receiving” something means it is obtained by any possible means, such as delivery of a physical item (for example, an array or array carrying package). When information is received it may be obtained as data as a result of a transmission (such as by electrical or optical signals over any communication channel of a type mentioned herein), or it may be obtained as electrical or optical signals from reading some other medium (such as a magnetic, optical, or solid state storage device) carrying the information. However, when information is received from a communication it is received as a result of a transmission of that information from elsewhere (local or remote).

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. Items of data are “linked” to one another in a memory when a same data input (for example, filename or directory name or search term) retrieves those items (in a same file or not) or an input of one or more of the linked items retrieves one or more of the others. In particular, when an array layout is “linked” with an identifier for that array, then an input of the identifier into a processor which accesses a memory carrying the linked array layout retrieves the array layout for that array.

A “computer”, “processor” or “processing unit” are used interchangeably and each references any hardware or hardware/software combination which can control components as required to execute recited steps. For example a computer, processor, or processor unit includes a general purpose digital microprocessor suitably programmed to perform all of the steps required of it, or any hardware or hardware/software combination which will perform those or equivalent steps. Programming may be accomplished, for example, from a computer readable medium carrying necessary program code (such as a portable storage medium) or by communication from a remote location (such as through a communication channel).

A “memory” or “memory unit” refers to any device which can store information for retrieval as signals by a processor, and may include magnetic or optical devices (such as a hard disk, floppy disk, CD, or DVD), or solid state memory devices (such as volatile or non-volatile RAM). A memory or memory unit may have more than one physical memory device of the same or different types (for example, a memory may have multiple memory devices such as multiple hard drives or multiple solid state memory devices or some combination of hard drives and solid state memory devices).

An array “assembly” includes a substrate and at least one chemical array on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

“Reading” signal data from an array refers to the detection of the signal data (such as by a detector) from the array. This data may be saved in a memory (whether for relatively short or longer terms).

A “package” is one or more items (such as an array assembly optionally with other items) all held together (such as by a common wrapping or protective cover or binding). Normally the common wrapping will also be a protective cover (such as a common wrapping or box) which will provide additional protection to items contained in the package from exposure to the external environment. In the case of just a single array assembly a package may be that array assembly with some protective covering over the array assembly (which protective cover may or may not be an additional part of the array unit itself.

It will also be appreciated that throughout the present application, that words such as “cover”, “base” “front”, “back”, “top”, “upper”, and “lower” are used in a relative sense only.

“May” refers to optionally.

When two or more items (for example, elements or processes) are referenced by an alternative “or”, this indicates that either could be present separately or any combination of them could be present together except where the presence of one necessarily excludes the other or others.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO4, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2 SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The phrase “plurality of ribonucleic acids” refers to a collection of two or more different ribonucleic acids of differing sequence. By plurality is meant at least 2, such as at least about 5, including at least about 10 different ribionucleic acids of differing sequence, where the number of distinct ribonucleic acids of differing sequence in a given plurality may be at least about 25, at least about 50, at least about 100, at least about 500, at least about 1000 or more, such as at least about 5,000 or more, at least about 10,000 or more, at least about 25,000 or more, etc.

The phrase a “single-stranded nucleic acid” refers to a first nucleic acid molecule that is not hybridized to a second nucleic acid, where the second nucleic acid molecule is not already covalently bound to the first nucleic acid. Thus, a single-stranded nucleic acid may be a linear molecule, where the linear molecule may or may not assume a secondary configuration such that a portion of the molecule is hybridized to itself, e.g., as in a hairpin configuration.

The phrase “RNA polymerase promoter domain” refers to a region or stretch of nucleotides having a sequence that is capable of initiating transcription of an operationally linked DNA sequence in the presence of ribonucleotides and an RNA polymerase under suitable conditions. The promoter domain may include between about 15 and about 250 nucleotides, such as between about 17 and about 60 nucleotides, from a naturally occurring RNA polymerase promoter or a consensus promoter region, as described in Alberts et al. (1989) in Molecular Biology of the Cell, 2d Ed. (Garland Publishing, Inc.). Prokaryotic promoters or eukaryotic promoters are of interest, and in representative embodiments prokaryotic promoters are employed, such as phage or virus promoters. As used herein, the term “operably linked” refers to a functional linkage between the affecting sequence (typically a promoter) and the controlled sequence, e.g., the variable domain as described below. The promoter regions that find use are regions where RNA polymerase binds tightly to the DNA and contain the start site and signal for RNA synthesis to begin. A wide variety of promoters are known and many are very well characterized. Representative promoter regions of interest include, but are not limited to: T7, T3 and SP6 as described in Chamberlin and Ryan, The Enzymes (ed. P. Boyer, Academic Press, New York) (1982) pp 87-108.

The phrase “variable domain” refers to a stretch or region of nucleic acids that has a sequence chosen to accomplish the particular application in which the array is to be used, and specifically the intended use of the ribonucleic acid mixture produced using the array in accordance with the subject methods. The length of the variable domain may vary considerably and will be chosen based on the desired length of the resultant ribonucleic acids in the to be produced RNA composition within the synthesis constraints of the subject method. In representative embodiments, the length of the variable domain will range from about 10 to about 150 nt, such as from about 15 to about 100 nt and including from about 20 to about 80 nt.

The phrase “linking domain” refers to an optional strecth or region of nucleotides between the promoter and the variable domain. If present, the linker domain may include between about 5 and 20 bases, but may be smaller or larger as desired. In representative embodiments, the linker domain, if present, has a length ranging from about 1 to about 20 bases, such as from about 1 to about 15 and including from about 1 to about 10, e.g., from about 5 to about 10 nt.

The term “spacer” refers an optional domain (i.e., stretch or region) located between the RNA polymerase promoter domain and the surface. The spacer domain, if present, may in representative embodiments have a length equivalent to the length of a nucleic acid sequence ranging froma bout 1 to about 25 nt, such as from about 5 to about 20 nt, including from about 5 to 15 nt. As mentioned above, the spacer is optional and may be any convenient sequence, including random sequence or a non-polynucleotide chemical linker (e.g. an ethylene glycol-based polyether oligomer), where a purpose of the spacer domain in certain embodiments is to project the other domains of the surface immobilized nucleic acids away from the substrate surface. In certain embodiments, the spacer domain is a polymer of monomeric residues chosen such that the spacer does not participate in Watson-Crick base pairing interactions, i.e., the spacer is non-hybridizable. Representative types of such spacers include, but are not limited to: polyethylene glycol spacers, polymers of abasic nucleotide residues, etc.

The phrase “nucleic acids complementary to an RNA polymerase promoter domain” refers to a collection (i.e., population) of oligonucleotides that have a sequence that is complementary to the sequence of an RNA polymerase promoter domain, such that oligonucleotides hybridize to the RNA polymerase promoter domain under stringent conditions.

The phrase “template array of overhang comprising nucleic acids” refers to an array having features made up of partially duplex nucleic acids, as described in greater detail below, where the overhang comprising nucleic acids include a double-stranded RNA polymerase promoter region and a single-stranded variable region overhang. The phrase “double-stranded RNA polymerase promoter region” refers to double-stranded stretch or region of base-paired nucleic acids made up of an RNA polymerase promoter domain and a nucleic acid complementary thereto that are hybridized to each other. The phrase “single-stranded variable region overhang” refes to a portion or stretch of a nucleic acid that is not hybridized to another nucleic acid and has a variable domain sequence.

The phrase “in vitro transcription protocol” refers to reaction conditions in which at least partially duplex DNAs are transcribed by an RNA polymerase to yield an RNA product. Such protocols are known in the art, see e.g. Milligan and Uhlenbeck (1989), Methods in Enzymol. 180, 51.

The phrase “primer extension reaction conditions” refers to reaction conditions that include contacting a primed nucleic acid in an aqueous reaction mixture with a source of DNA polymerase, dNTPs and any other desired or requisite primer extension reagents under conditions sufficient to produce the desired surface immobilized duplex nucleic acids, as further described below.

The term “separating” refers to physically dividing two initially combined entities.

The term “label” refers to a detectable moiety or agent. Labels of interest include directly detectable and indirectly detectable radioactive or non-radioactive labels such as fluorescent dyes. Directly detectable labels are those labels that provide a directly detectable signal without interaction with one or more additional chemical agents. Examples of directly detectable labels include fluorescent labels. Indirectly detectable labels are those labels which interact with one or more additional members to provide a detectable signal. In this latter embodiment, the label is a member of a signal producing system that includes two or more chemical agents that work together to provide the detectable signal. Examples of indirectly detectable labels include biotin or digoxigenin, which can be detected by a suitable antibody coupled to a fluorochrome or enzyme, such as alkaline phosphatase. In many preferred embodiments, the label is a directly detectable label. Directly detectable labels of particular interest include fluorescent labels. Fluorescent labels that find use in the subject invention include a fluorophore moiety. Specific fluorescent dyes of interest include: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl] benzoic acid ethyl ester monohydrochloride (R6G)(emits a response radiation in the wavelength that ranges from about 500 to 560 nm), 1, 1, 3, 3, 3′, 3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, and the like.

### DESCRIPTION OF THE SPECIFIC EMBODIMENTS

Methods and compositions for generating a plurality of distinct ribonucleic acids are provided. In the subject methods, an array is employed as a template in an in vitro transcription reaction. A feature of the template arrays employed in the subject methods is that they include a plurality of distinct features of surface immobilized nucleic acids that include a surface proximal RNA polymerase promoter domain and a surface distal variable domain. Also provided are the arrays employed in the subject methods and kits for practicing the subject methods. The ribonucleic acids produced by the subject methods find use in a variety of different applications, including differential gene expression analysis and gene-silencing applications.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless th context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Methods recited herein may be carried out in any order of the recited events which is logically possible, as well as the recited order of events.

All patents and other references cited in this application, are incorporated into this application by reference except insofar as they may conflict with those of the present application (in which case the present application prevails). The citation of any publication is for its disclosure prior to the filing date and should not be construed as an admission that the present invention is not entitled to antedate such publication by virtue of prior invention.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. The figures shown herein are not necessarily drawn to scale, with some components and features being exaggerated for clarity.

Methods

As summarized above, the subject invention provides array-based methods for generating or producing pluralities of distinct ribonucleic acids. By plurality is meant at least 2, such as at least 5, including at least 10, where the number of distinct product nucleic acids in the plurality may be at least about 25, at least about 50, at least about 100, at least about 500, at least about 1000 or more, such as at least about 5,000, at least about 10,000, at least about 25,000 or more, including but not limited to 30,000 or more, 50,000 or more, 100,000 or more, 250,000 or more, 400,000 or more, etc. The product ribonucleic acids may be heterogeneous mixtures, or a collection of individual homogeneous populations, as described in greater detail below.

The subject methods of producing the above-described pluralities are array-based methods, where a feature of the subject methods is that a nucleic acid array is employed as template. In practicing the subject methods, the first step is generally to contact an initial precursor nucleic acid array with a nucleic acids complementary to an RNA polymerase promoter domain (i.e., an RNA polymerase promoter complement composition) under conditions sufficient to produce a template array of overhang containing duplex nucleic acids. The resultant template array is then employed in the second step of the subject methods to produce a product plurality of ribonucleic acids. Each of these steps is now described separately in greater detail.

The initial array employed in the first step of the subject methods, which may conveniently be referred to as the template array generation step, is (in representative embodiments) a substrate having a planar surface on which is immobilized a plurality of distinct nucleic acid features of surface immobilized nucleic acids. (As is known in the art, the array may also be present as a “fluid” array made up of a plurality of different beads or analogous structures, each of which bears an immobilized nucleic acid and serves as a “region” of the array.) The surface immobilized nucleic acids of a given feature on the array are made up of single-stranded nucleic acids, and in many embodiments single-stranded deoxyribonucleic acids (where a single-stranded nucleic acid is a nucleic acid that is not hybridized to a second, non-covalently bound nucleic acid). The surface immobilized single-stranded nucleic acids include a RNA polymerase promoter domain and a variable domain. The initial arrays employed in the subject methods may be generated de novo or obtained as a pre-made array from a commercial source, where in either case the array will have the characteristics described below. Arrays of nucleic acids are known in the art, where representative arrays that may be modified to become arrays of the subject invention as described below, include those described in: U.S. Pat. Nos. 6,656,740; 6,613,893; 6,599,693; 6,589,739; 6,587,579; 6,420,180; 6,387,636; 6,309,875; 6,232,072; 6,221,653; and 6,180,351 and the references cited therein.

The number of nucleic acid features of the initial or precursor array may vary, where the number of features present on the surface of the array may be at least 2, 5, or 10 or more such as at least 20 and including at least 50, where the number may be as high as about 100, as about 500, as about 1000, as about 5000, as about 10000 or higher, e.g., 25,000 or higher, 50,000 or higher, 100,000 or higher, 500,000 or higher, 1,000,000 or higher, etc. In representative embodiments, the subject arrays have a density ranging from about 1000 to about 10,000 features/cm², such as from about 2,000 to about 10,000 features/cm², including from about 2,000 to about 5,000 features/cm². In representative embodiments, the density of single-stranded nucleic acids within a given feature is selected to optimize efficiency of the RNA polymerase. In certain of these representative embodiments, the density of the single-stranded nucleic acids may range from about 10⁻³ to about 1 pmol/mm², such as from about 10⁻² to about 0.1 pmol/mm², including from about 5×10⁻² to about 0.1 pmol/mm².

As mentioned above, each distinct surface immobilized nucleic acid of the features on the array includes an RNA polymerase promoter domain and a variable domain. In representative embodiments, the RNA polymerase promoter domain is positioned closer to the surface than the variable domain, such that the RNA polymerase promoter domain may be viewed as a surface proximal RNA polymerase promoter domain and the variable domain may be viewed as a surface distal variable domain. In those embodiments where the surface immobilized nucleic acids are immobilized to the surface by their 3′ ends, the RNA polymerase promoter domain is typically 3′ of the variable domain in the nucleic acid, such that it is located at the 3′ end of the nucleic acid and the variable domain is located at the 5′ end of the nucleic acid.

The variable domains of the features of the precursor array have sequences that are chosen based on the particular application in which the array is to be used, and specifically the intended use of the ribonucleic acid mixture produced using the array in accordance with the subject methods. The length of the variable domain may vary considerably and will be chosen based on the desired length of the resultant ribonucleic acids in the to be produced RNA composition within the synthesis constraints of the subject method. In representative embodiments, the length of the variable domain will range from about 10 to about 150 nt, such as from about 15 to about 100 nt and including from about 20 to about 80 nt.

As mentioned above, in addition to the variable domain, each surface immobilized nucleic acid present on the array includes an RNA polymerase promoter domain, which domain may be the same or different between or among the features of the array. Where different RNA polymerase promoter domains are represented in the feature population of the array, the number of different RNA polymerase promoter domains does not exceed about 5, and does not exceed about 3 in certain embodiments. In representative embodiments, a single RNA polymerase promoter domain is represented or present in all of the features of the array, such that the RNA polymerase promoter domain is common or the same among all of the nucleic acids of all of the features of the array. Suitable polymerase promoter domains that find use in the subject methods are ones that are capable of initiating transcription of an operationally linked DNA sequence in the presence of ribonucleotides and an RNA polymerase under suitable conditions. The promoter domain is linked in an orientation to permit transcription of RNA, as described in greater detail below. The promoter region may include between about 15 and about 250 nucleotides, such as between about 17 and about 60 nucleotides, from a naturally occurring RNA polymerase promoter or a consensus promoter region, as described in Alberts et al. (1989) in Molecular Biology of the Cell, 2d Ed. (Garland Publishing, Inc.). Prokaryotic promoters or eukaryotic promoters may be employed, and in representative embodiments prokaryotic promoters are employed, such as phage or virus promoters. As used herein, the term “operably linked” refers to a functional linkage between the affecting sequence (typically a promoter) and the controlled sequence, e.g., the variable domain. The promoter regions that find use are regions where RNA polymerase binds tightly to the DNA and contain the start site and signal for RNA synthesis to begin. A wide variety of promoters are known and many are very well characterized. Representative promoter regions of interest include, but are not limited to: T7, T3 and SP6 as described in Chamberlin and Ryan, The Enzymes (ed. P. Boyer, Academic Press, New York) (1982) pp 87-108.

In certain embodiments, the surface immobilized nucleic acids of the features of the array further include a spacer domain located between the RNA polymerase promoter domain and the surface. The spacer domain, if present, may in representative embodiments have a length equivalent to the length of a nucleic acid sequence ranging froma bout 1 to about 25 nt, such as from about 5 to about 20 nt, including from about 5 to 15 nt. As mentioned above, the spacer domain is optional and may be any convenient sequence, including random sequence or a non-polynucleotide chemical linker (e.g. an ethylene glycol-based polyether oligomer), where a purpose of the spacer domain in certain embodiments is to project the other domains of the surface immobilized nucleic acids away from the substrate surface. In certain embodiments, the spacer domain is a polymer of monomeric residues chosen such that the polymeric spacer does not participate in Watson-Crick base pairing interactions, i.e., the spacer is non-hybridizable. Representative types of such spacers include, but are not limited to: polyethylene glycol spacers, polymers of abasic nucleotide residues, etc.

In certain embodiments, a linker domain between the promoter and the variable domain may be present. If present, the linker domain may include between about 5 and 20 bases, but may be smaller or larger as desired. In representative embodiments, the linker domain, if present, has a length ranging from about 1 to about 20 bases, such as from about 1 to about 15 and including from about 1 to about 10, e.g., from about 5 to about 10 nt. In certain embodiments, the linker domain may be cleavable, e.g., have a nuclease recognized sequence of nucleotides.

In representative embodiments, each surface immobilized nucleic acid on the array employed in the subject methods is described by the following formula:
surface-Sₛ-R-L_{I}-V-5′
wherein:
   S is a spacer;
   s is an integer of 0 or 1;
   R is said surface proximal RNA polymerase promoter domain;
   L is a linker domain;
   I is an integer of 0 or 1; and
   V is said surface distal variable domain;
   where each of the above features is as described above

In certain of these representative embodiments, only the variable domain V of said surface immobilized single-stranded nucleic acids differs between features.

The subject arrays are provided by any convenient means, including obtaining them from a commercial source or by synthesizing them de novo. To synthesize the arrays employed in the subject methods, the first step is generally to determine the nature of the mixture of nucleic acids that is to be produced using the subject array according to the subject methods. For example, in those embodiments where the nucleic acid mixture is to be employed as a reference or control in a differential gene expression application, as described in greater detail below, the first step is to identify those genes that are to be assayed in the particular protocol to be performed. Following identification of these genes, the specific region, i.e., stretch or domain, of each product RNA to which the probe nucleic acid is to hybridize is then identified. Any convenient method may be employed to determine the sequences of the surface immobilized nucleic acids, including probe design algorithms, including but not limited to those algorithms described in U.S. Pat. No. 6,251,588 and published U.S. Application Nos. 20040101846; 20040101845; 20040086880; 20040009484; 20040002070; 20030162183 and 20030054346; the disclosures of which are herein incorporated by reference. Following identification of the probe sequences as defined above, an array is produced in which each of the probe sequences of the identified set is present.

Following provision of the array employed in the subject methods, as described above, the next step is to contact the array with a RNA polymerase promoter complement composition (i.e., nucleic acids complementary to a RNA polymerase promoter) under hybridization conditions sufficient to produce a template array that includes a plurality of overhang comprising duplex nucleic acids on its surface, where the overhang is made up of the variable domain of each surface immobilized nucleic acid of the initial or precursor array. The RNA polymerase promoter complement composition is a nucleic acid composition that is made up of one or more distinct types of nucleic acids of different sequence, where a given nucleic acid member of the complement composition is capable of hybridizing to an RNA polymerase promoter domain present on the array. The complement composition may be homogeneous or heterogenous, depending on whether there is a single RNA polymerase promoter domain represented on the array, or a plurality of different such promoter domains. The nucleic acid members of the complement composition have a length that is sufficient to bind to the complementary domain on the array and produce a functional RNA polymerase promoter site, where the length of the constituent nucleic acid members may range from about 10 to about 45 nt, such as from about 15 to about 35 nt and including from about 20 to about 30 nt.

As mentioned above, the template array produced by this method is an array of double-stranded (i.e., duplex)nucleic acids made up of a first nucleic acid having a polymerase promoter and complement variable domains and a second nucleic acid which is hybridized to the polymerase promoter domain. As such, the array produced by this step is an array of overhang comprising duplex nucleic acid molecules, where the overhang is made up of the variable domain of each probe on the array.

Optionally, the resultant template array may be subjected to primer extension reaction conditions sufficient to produce an array of surface immobilized full-length duplex nucleic acids, conveniently referred to herein as a template array of full-length duplex nucleic acids. The specific primer extension reaction conditions to which the template array of overhang comprising duplex nucleic acids is subjected may vary, so long as the conditions produce the desired surface immobilized duplex nucleic acids. In representative embodiments, the array is contacted in an aqueous reaction mixture with a source of DNA polymerase, dNTPs and any other desired or requisite primer extension reagents under conditions sufficient to produce the desired surface immobilized duplex nucleic acids. The polymerase employed in this optional step of the subject methods may or may not be a thermostable polymerase. A variety of thermostable polymerases are known to those of skill in the art, where representative polymerases include, but are not limited to: Taq polymerase, Vent® polymerase, Pfu polymerase and the like. The amount of polymerase present in the reaction mixture may vary but is sufficient to provide for the requisite amount of polymerase activity, where the specific amount employed may be readily determined by those of skill in the art. Also present in the reaction mixture is a collection of the four dNTPs, i.e., dATP, dCTP, dGTP and dTTP. The dNTPs may be present in varying or equimolar amounts, where the amount of each dNTP typically ranges from about 10 μM to 10 mM, usually from about 100 μM to 300 μM. Other reagents that may be present in the reaction mixture include: monovalent cations (e.g. Na⁺), divalent cations (e.g. Mg⁺⁺), buffers (e.g. Tris), surfactants (e.g. Triton X-100) and the like. The reaction mixture is maintained at a suitable primer extension temperature for a suitable period of time, where in representative embodiments, the primer extension temperature ranges from about 55° C. to 75° C., usually from about 60° C. to 70° C. and is maintained for period of time ranging from about 30 sec. to 10 min., such as from about 1 min. to 5 min.

Following production of the template arrays, e.g., overhang or optional duplex template arrays, as described above, the resultant template array is then subjected to in vitro transcription reaction conditions sufficient to produce the desired product ribonucleic acid plurality. During this step, the at least partially duplex DNAs produced in the first step of the methods are transcribed by RNA polymerase to yield RNA product. In this step, the at least partially duplex DNAs are contacted with the appropriate RNA polymerase in the presence of the four ribonucleotides, under conditions sufficient for RNA transcription to occur, where the particular polymerase employed will be chosen based on the promoter region present in the double-stranded DNA, e.g. T7 RNA polymerase, T3 or SP6 RNA polymerases, E. coli RNA polymerase, and the like. Suitable conditions for RNA transcription using RNA polymerases are known in the art, see e.g. Milligan and Uhlenbeck (1989), Methods in Enzymol. 180, 51.

Where desired, the RNA pluralities that are produced by the subject methods may be produced as labeled pluralities of RNAs. The label may be incorporated into the product RNAs using any convenient protocol, e.g., by employing labeled NTPs in the in vitro transcription reaction mixture, or by employing labeled RNA polymerase promoter complements. Further details regarding representative labels and manners of using the same are provided above.

The above-described methods result in the production of a plurality of ribonucleic acids, where each of the different variable domains of the template array is represented in the plurality, i.e., for each feature present on the template array, there is at least one ribonucleic acid in the plurality that corresponds to the feature, where by corresponds is meant that the nucleic acid is one that is generated by in vitro transcription using the variable domain of the feature as template. The length of each of the product ribonucleic acids present in the resultant plurality ranges, in representative embodiments, from about 20 to about 500 nt or longer, such as from about 50 to about 200 nt, including from about 60 to about 100 nt. The plurality of ribonucleic acids produced in these embodiments of the subject methods is characterized by having a known composition. By known composition is meant that, because of the way in which the plurality is produced, the sequence of each distinct ribonucleic acid in the product plurality can be predicted with a high degree of confidence. Accordingly, assuming no infidelities occur in the polymerase mediated ribonucleic acid generation step of the subject methods, the sequence of each individual or distinct nucleic acid in the product plurality is known. In many embodiments, the relative amount or copy number of each distinct ribonucleic acid of differing sequence in the plurality is known. Put another way, the product plurality of ribonucleic acids is known to include a constituent ribonucleic acid corresponding to each feature of the template array used to produce it, such that each feature of the template array is represented in the product plurality.

A feature of the ribonucleic acids of the product pluralities is that they are single-stranded ribonucleic acids. As such, the ribonucleic acids of the subject pluralities are not hybridized to complementary ribonucleic acids. In other words, the constituent ribonucleic acids of the product pluralities are not hybridized to separate ribonucleic acids of complementary sequence, where the separate ribonucleic acids are not covalently joined to them. While the product ribonucleic acids of the plurality are single-stranded, they may be linear or assume some secondary configuration, e.g., a hairpin configuration, and the like. The number of different or distinct ribonucleic acids present in the product plurality may vary, but is generally at least 2, at least 5, at least 10, such as at least about 20, at least about 50, at least about 100 or more, where the number may be as great as about 1000, about 5000 or about 25,000 or greater. Any two given RNAs in the product pluralities are considered distinct or different if they include a stretch of at least 20 nucleotides in length in which the sequence similarity is less then 98%, as determined using the FASTA program (using default settings).

As indicated above, the product plurality of ribonucleic acids may be a heterogenous mixture or set of individual homogeneous RNA compositions, depending on the intended use of the product plurality.

For those embodiments where the product plurality is a mixture, the term mixture refers to a heterogenous composition of a plurality of different ribonucleic acids that differ from each other by residue sequence. Accordingly, the mixtures produced by the subject methods may be viewed as compositions of two or more ribonucleic acids that are not chemically combined with each other and are capable of being separated, e.g., by using an array of complementary surface immobilized nucleic acids, but are not in fact separated.

In those embodiments where the plurality of ribonucleic acids is a set of homogenous ribonucleic acid populations, the constituent members of the set are, in certain embodiments, physically separated, such as present on different locations of a solid support, present in different containment structures, and the like.

In certain embodiments, the product pluralities of ribonucleic acids are physically separated from the template array used in the production thereof. In yet other embodiments, the product pluralities may be associated with the template array, e.g., present on the features of the template array.

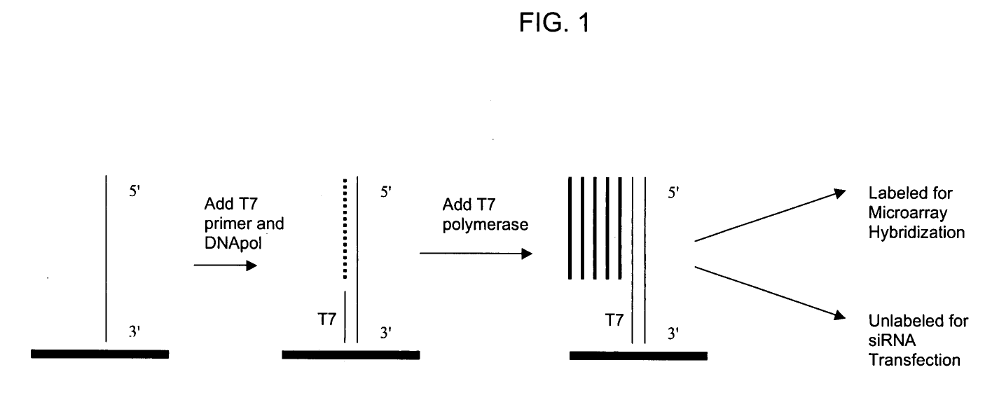

FIG. 1 provides a schematic view of a representative embodiment of the subject methods. In FIG. 1, a nucleic acid of a feature of an array is shown as a vertical line attached to an array substrate surface at its 3′ end. In the first step of the depicted representative embodiment, T7 primer and DNA polymerase, as well as dNTPs and other primer extension reagents, e.g., buffer, etc. as reviewed elsewhere herein, are conacted with the substrate under primer extension conditions to produce a template array of double stranded nucleic acids. In the next step, the resultant template array is contacted with T7 polymerase, NTPs and other in vitro description reagents, as reviewed above, to produce multiple RNA transcripts. Depending on the intended use of the transcripts, the transcripts may be labeled or unlabeled.

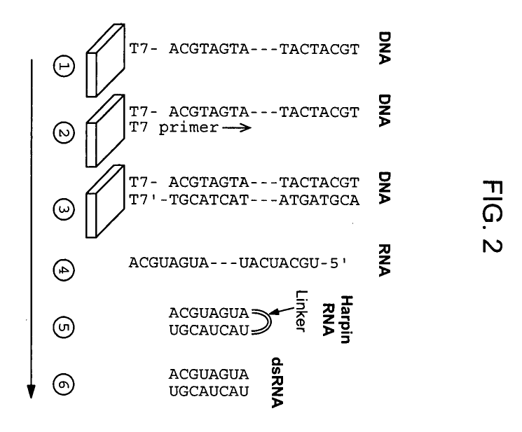

FIG. 2 provides a schematic view of an alternative representative embodiment of the subject methods. FIG. 2 shows the progression of DNA synthesis on arrays and is representative of a particular sequence represented on a particular addressable feature. In step 1, a 3′-T7 complement sequence is printed followed by a sequence of a nulceic acid of interest. A T7 primer is then hybridized to the surface immobilized sequence, followed by primer dependent extension to produce a double stranded structure as depicted in step 3. Upon in vitro transcription, a sequence represented in step 4 is obtained that reads from 5′-UGCAUCAU-linker-AUGAUGCA. The linker assists in fold back and can be removed enzymatically or by using nucleases, as desired, e.g., to produce siRNA duplexes.

The product pluralities of ribonucleic acids find use in a variety of different applications, representative applications of which are reviewed in greater detail in the following section.

Utility

The pluralities of ribonucleic acids produced by the subject methods find use in a variety of different applications, where two representative types of applications that are described in greater detail below are gene expression applications and gene-silencing applications.

Gene Expression Applications

Gene expression analysis protocols are well known to those of skill in the art, and therefore need not be reviewed in great detail. In gene expression analysis protocols, a population of target nucleic acids (which may be labeled) is contacted with a population of probe nucleic acids, e.g., immobilized on a surface of a solid support, e.g., in the form of an array, under hybridization conditions, such as stringent hybridization conditions. Following hybridization, non-bound target is removed or separated from the probe, e.g., by washing. Washing results in a pattern of hybridized target, which may be read using any convenient protocol, e.g., with a fluorescent scanner device where fluorescent labels are employed. From this pattern, information regarding the mRNA expression profile in the initial mRNA sample from which the target population was produced may be readily derived or deduced.

Use of RNA Pluralities as a Control or Reference

In gene expression analysis applications, the RNA pluralities produced by the subject invention may find use, in certain embodiments, as control sets of target nucleic acids, where at least a subset of the probe nucleic acids employed in the assay, and in certain embodiments all of the probe nucleic acids employed in the assay and present on the array, are represented in the control set. In other words, the control set includes a nucleic acid capable of hybridizing to each different probe nucleic acid of at least a subset of all of the different probe nucleic acids of the array with which it is employed.

In those embodiments where the product RNA pluralities are employed as controls or references for a gene expression assay, the control set of target nucleic acids produced by the subject methods may include at least one target nucleic acid complementary to each probe nucleic acid present in at least a subset of the probe nucleic acids present on the array with which they are used. In other words, at least a subset of the probe nucleic acids present on a given array are represented in the control set intended for use with the given array. In representative embodiments, by at least a subset is meant that at least 20, usually at least 30 and more usually at least 50 of the probe nucleic acids present on the array are represented in the control set. In certain embodiments, at least 20%, usually at least 30% and more usually at least 50% of the probe nucleic acids present on the array are represented in the array. In representative embodiments, all of the probe nucleic acids present on the array are represented in the control set. For example, where a given array includes 500 distinct probe nucleic acids which are distinct from each other based on sequence, a control set for use with this particular array includes at least 500 different target nucleic acids—one for each probe nucleic acid on the array.

Non-probe sequences on the array may not have a target nucleic acid in the control set, e.g., array sequences such as orientation sequences, negative and positive control sequences, etc. that may be present on an array. In general, control target nucleic acids are not necessary for sequences on an array that do not require quantification, where a particular protocol is intended to provide qualification data only.

The number of unique control target nucleic acids in the set or pool of control target nucleic acids will, in representative embodiments, be at least about 20, usually at least about 50, more usually at least about 100, where the number may be as high as 1000 or higher.

Control target nucleic acids can be the same length, shorter or longer than their corresponding probe sequences on the array or test nucleic acid in the solution (if present). However, each control target nucleic acid may be designed to have a least partial complementarity to its corresponding probe nucleic acid and at least partial sequence identity with its corresponding test target nucleic acids (if present). In general, the length of each target nucleic acid in a given control set designed for such uses is at least about 25 nucleotides, such as at least about 50 nucleotides, including at least about 100 nucleotides or longer. In addition, the control target nucleic may be designed to have structural and hybridization characteristics very similar to its corresponding test target nucleic acid, i.e., it may be designed to have similar hybridization efficiencies, similar kinetics with complementary probe sequences, similar background hybridization with other sequences, etc.

A feature of control sets of target nucleic acids is that the concentration of each control target nucleic acid present in the set is known, where such feature is provided by using the subject methods to prepare the control sets. As such, by selecting the appropriate features and numbers thereof, as well as the conditions of in vitro transcription, the composition of the product RNA plurality that is subsequently used as a control or reference may be tailored as desired, and therefore known.

Depending on the particular assay protocol with which the control sets of target nucleic acids are employed, the control and test (denoting the target nucleic acids prepared from the sample being assayed in a given protocol) sets of target nucleic acids may be labeled with the same label, such that the test and control sets cannot be distinguished from one another, or the test and control sets of target nucleic acids may be differentially labeled, such that the two sets are readily distinguishable from each other.

As such, in certain embodiments, the test and control sets of target nucleic acids are differentially labeled. By “differentially labeled” is meant that the test and control sets of target nucleic acids are labeled differently from each other such that they can be simultaneously distinguished from each other. For example, where one has a control set of target nucleic acids and test set of target nucleic acids, each target nucleic acid in the test set will be labeled with the same first label and each target nucleic acid in the control set will be labeled with the same second label that is different and distinguishable from the first label. Likewise, where two control sets are employed in the method, each target nucleic acid in the second control set will be labeled with a third label different and distinguishable from both the first and second label.

In yet other embodiments, the test and control sets of target nucleic acids are labeled with the same label, so as to be indistinguishable from each other. When the test and control sets of target nucleic acids are labeled with the same label, each target nucleic acid of each set is labeled with the same label.

A variety of different labels may be employed, where such labels include fluorescent labels, isotopic labels, enzymatic labels, particulate labels, etc, as described above. Any combination of labels, e.g. first and second labels, first, second and third labels, etc., may be employed for the test and control target sets, provided the labels are distinguishable from one another. Examples of distinguishable labels are well known in the art and include: two or more different emission wavelength fluorescent dyes, like Cy3 and Cy5, or Alexa 542 and Bodipy 630/650; two or more isotopes with different energy of emission, like ³²P and ³³P; labels which generate signals under different treatment conditions, like temperature, pH, treatment by additional chemical agents, etc.; and labels which generate signals at different time points after treatment. Using one or more enzymes for signal generation allows for the use of an even greater variety of distinguishable labels based on different substrate specificity of enzymes (e.g. alkaline phosphatase/peroxidase).

In use, the test and control sets of target nucleic acids may be hybridized to an array, where the sets of target nucleic acids may be hybridized to the same array or different arrays, where when the sets of target nucleic acids are hybridized to different arrays, all of the different arrays will at least share common arrays of probe nucleic acids, i.e., they will be identical with respect to their probe nucleic acids.

In certain embodiments, the test and control sets of target nucleic acids are hybridized to the same array. In such embodiments, the array is hybridized with a test set of labeled target nucleic acids and at least one control set of labeled target nucleic acids. In those embodiments where more than one control set of target nucleic acids is employed, the number of different control sets may range from 2 to 6, usually 2 to 4 and more usually 2 to 3. Of particular interest are those embodiments in which 1 or 2 different control sets of target nucleic acids are employed.

The test and control sets of target nucleic acids may be hybridized to the array and/or detected simultaneously or sequentially. Thus, where a control set and test set are employed, the two sets of target nucleic acids may be combined prior to hybridization and the array hybridized to both simultaneously to minimize potential variability in hybridization conditions. For example, a known amount of labeled sets of test target and control target nucleic acids can be added to the same hybridization buffer, and then contacted with one or more arrays simultaneously under hybridization conditions. In another example, a known amount of labeled sets of test target and control target nucleic acids are added to the same hybridization mix, and this buffer aliquoted for the separate hybridization of different arrays. By storing aliquots of the hybridization mix (e.g. storage at −20° C. or −70° C.), different arrays may be hybridized at different times with approximately the same amounts of target nucleic acid sequences.

In the above embodiments where the test and control target nucleic acids are hybridized simultaneously to a given array, labeled test and control target nucleic acids may be premixed or pooled prior to contact with the array. In representative embodiments, mixtures of test and control target nucleic acids have amounts of control and target nucleic acids which are sufficient to generate signals that are at least 10 fold, usually at least 20 fold and more usually at least 50 fold higher than background signals observed with the array. The relative amounts of control and test target nucleic acids in the mixture are selected to be sufficient to allow reliable detection of the test sequences complimentary to the probe nucleic acid while at the same time allowing complete binding of the test target nucleic acids with a nofold excess of unbound probe nucleic acid on the array.

Alternatively, one or more arrays may be hybridized with the control and test sets of target nucleic acids sequentially. For example, arrays may be hybridized with a hybridization mix containing the labeled test target nucleic acids to allow these molecules uninhibited access to the probe sequences of the array. Following this hybridization, control target nucleic acids could then be exposed to the array for use as an internal control. The hybridization of the control target nucleic acids may be completely separate from the hybridization of the test target nucleic acids, e.g. using different hybridization mixes at different times, or the control target sequences may be added to the hybridization buffer containing the test target nucleic acids following an incubation period with the test target nucleic acids. When used sequentially, the control and test target nucleic acids may be differentially labeled or labeled with the same label, since detection occurs separately.

In yet other embodiments, the test and control sets of target nucleic acids may be hybridized to different arrays, where each of the different arrays has an identical population of probe sequences, i.e. the different arrays do not vary with respect to their probe sequences. In such methods, the control and test target nucleic acids may be labeled with the same label so as to be indistinguishable from one another, and discussed above.

Following hybridization, non-hybridized labeled nucleic acid is removed from the support surface, conveniently by washing, generating a pattern of hybridized nucleic acid on the substrate surface. A variety of wash solutions and protocols are known to those of skill in the art and may be used. See the representative conditions provided above.

The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, colorimetric measurement, light emission measurement, light scattering and the like.

Following detection or visualization, the hybridization patterns generated by control and test target nucleic acids may be compared to identify differences between the signals. Where arrays in which each of the different probes corresponds to a known gene are employed, differences in signal intensity can be related to a different target concentration of a particular gene. The comparison of the intensity of binding of a test target nucleic acid to a probe sequence can be compared to the intensity of the binding of the corresponding control target sequence, and the measurement converted to a quantitative RNA concentration for that target sample. The quantitative RNA levels of the test target can be compared between arrays to identify or confirm differential expression of genes in particular samples.

By using RNA pluralities produced via the subject methods as control or reference target nucleic acids, as reviewed above, a number of different tasks can be accomplished, which tasks include, but are not limited to: detecting relative hybridization of target sequences, calibrating a hybridization assay, harmonizing data between hybridization assays, and testing reagents used in a hybridization assay.

Control or reference sets of target nucleic acids are useful in detecting relative levels of hybridization of different genes in a sample by providing a set of internal hybridization controls. Since the control set of nucleic acids are of a known sequence, in a known quantity, and of a known specific activity (where in a preferred embodiment the control and test target are labeled with the same specific activity), the level of hybridization of the control nucleic acids can be used to determine the level of expression of each gene in a test sample based on its level of binding to a probe sequence. The fact that each probe sequence has its own internal control also allows for the detection of potential expression differences between samples and differences in binding affinities between probe sequences, both on a single array and between arrays. Thus, the intensity level of hybridization of a control sequence can be used to calculate the expression level of a gene in a sample based upon the intensity of the test target hybridization to the corresponding probe sequence.

Use of the subject RNA pluralities as control or reference sets of target nucleic acids also finds use in the calibration of hybridization assays. Using known concentrations of probe nucleic acid, test target nucleic acids, and control target nucleic acids allows one to optimize the hybridization conditions for a particular use, such as increasing stringency to allow better detection of nucleic acids with some level of sequence homology (e.g. differential expression between genes from a single family or alternative splice forms for the same gene). The use of the internal standards of the method of the subject invention allows hybridization, labeling procedures, and the like to be optimized for a particular use, which is especially valuable for standardization of large scale of hybridization assays, such as high-throughput screening of biological samples. Optimization thus means that one can change hybridization conditions in order to achieve maximal intensity of specific hybridization signals with complimentary probe sequences and minimal level of non-specific hybridization with non-complementary probe sequences.

Use of the RNA pluralities of the present invention as control or reference sets also provides for the opportunity to harmonize data between hybridization assays, thus allowing for a direct comparison of expression levels despite potential differences due to variables such as differences in hybridization conditions, differences in sample preparation and even between different types of arrays, differences in quality and performance within and between different arrays, differences in specific activity of the labeled target sequences, and the like. Because each hybridization assay has its internal control for at least a subset of the probe sequences on the array, the data can be compared using ratios of the intensity of the control target nucleic acids and the intensity of the test target nucleic acids. Thus, the use of simple mathematical formulations to correct for differences between assays allows the levels of gene expression in these different assays to be adjusted to the same level and then compared in a biologically relevant fashion.

Control or references sets of target nucleic acids that are prepared by the subject methods are also useful in determining the efficacy of hybridization reagents. Such reagents may be, for example, new reagents, e.g., different buffer solutions for prehybridization and hybridization, or established reagents, e.g., a new batch of a known, commercially available reagent. The internal control of the methods of the subject invention provide for two levels of quality assurance upon testing the reagents, basically providing an extra control for determining the efficacy of a reagent in a single hybridization. Efficiency means maximum specific signal with minimal level of non-specific signal and background binding to solid surface. Other parameters such as temperature, buffer composition, length of hybridization and/washing times, etc., may be optimized using calibration controls. Also, the same calibration target nucleic acids can be used routinely to test and calibrate detection equipment to expected level intensity of signals, thus limiting variability due to functionality of the equipment; and may be used to test and calibrate the quality of arrays for control procedures.

Use of RNA Pluralities to Estimate/Correct for Noise and/or Cross-Hybridization in a Single Color Array Assay

The RNA product mixtures also find use as control target mixtures in the estimation and or correction of noise and/or cross-hybridization in single color array assays. For example, with the subject methods one can produce defined mixtures of ribonucleic acids that include target nucleic acids having sequences of known mismatch with respect to sequences present in the probes of an array, and use the signals obtained from such mismatch target nucleic acids to estimate and/or correct of noise, as is known in the art. For example, complex RNA target mixtures can be produced using the subject methods that include RNAs that are, with respect to the probes of the array with which the complex mixture is used: (1) perfect matches; (2) mismatches of greater than 2 to 5 bases (by sequence inversion); (3) deletions; and (4) random. The signals obtained from using such a mixture may then be employed in conjunction with appropriate algorithms to estimate noise in a given single color experiment, and correct therefor. RNA mixtures can also be produced by the subject methods that include a plurality, e.g., 2 to 7, of different targets of varying degrees of mismatch for a given probe nucleic acid, and used to provide an estimation of non-specific signal and sensitivity. Such mixtures can be employed as QC metrics. The subject methods may also be employed to produce RNA mixtures that can be employed for determining RNA quality.

In certain embodiments, a RNA target mixture is produced by the subject methods that is designed to specifically bind to the probes present on the array, or alternatively is not designed to specifically bind to probes present on the array. The former type of mixture may be employed to model or estimate the specific signal obtained from the array, while the latter type of mixture may be employed to model or estimate the non-specific signal obtained from the array. When a different signal channel is employed from the test target signal channel, estimation of the specific or non-specific signal avoids signal contamination which may originate in the test signal channel. By estimating a noise or non-specific signal value in this manner (and if desired reconstructing this signal value into the experimental channel, e.g., to compensate for differences in intensities between signal, e.g., green and red, channels) and then removing the noise signal from the raw intensity signal, true intensity of a signal can be imputed. This true intensity signal better correlates with the transcript concentration in a sample than unadjusted raw or normalized data.

Gene-Silencing Applications

The subject methods of producing pluralities of ribonucleic acids also find use in gene-silencing applications. For example, the array-based methods of producing pluralities of ribonucleic acids find use in producing RNAi agents, such as short hairpin RNA molecules.

In such applications, the variable sequences of the template arrays are chosen to encode RNAi molecules, e.g., shRNA molecules. The template array may be designed to produce RNAi molecules to a variety of different genes, or a plurality of different RNA molecules designed to silence the same gene. The template array may be configured as an array of subarrays (including a multiwell format, e.g., 8 well, 96 well, 384 well etc.), where such configurations are known in the art, where each subarray has features designed to produce siRNA molecules to a different gene.

When the subject methods are employed to produce siRNA molecules, the RNA molecules of the product pluralities are typically not labeled. In certain embodiments, the product siRNA molecules may not be separated from the arrays prior to use in gene silencing experiments, where the resultant template arrays that include unbound product siRNA molecules in each feature following the in vitro transcription step are employed as siRNA arrays, e.g., as described in Published U.S. Patent Applications Nos. 20030228694; 20030228601; 20030203486 and 20020006664; the disclosures of which are herein incorporated by reference. Alternatively, the product pluralities may be separated from the template array used to produce them, and then subsequently used in RNAi mediated gene silencing applications.

It is noted that the above reviewed applications are merely representative of the different applications in which the product RNA pluralities produced by the methods of the subject invention find use.

Data Transmission Embodiments

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. The data may be transmitted to the remote location for further evaluation and/or use, whereupon arrival at the remote location, it may be received by a user. Any convenient telecommunications means may be employed for transmitting the data, e.g., facsimile, modem, internet, etc.

Kits

Also provided by the subject invention are kits for use in preparing the subject target populations of nucleic acids. The kits may comprise containers, each with one or more of the various reagents (typically in concentrated form) utilized in the methods, including, for example, buffers, dNTPs, reverse transcriptase, etc., where the kits will at least include a sufficient amount of RNA polymerase promoter domain complementary nucleic acids, e.g., an amount ranging from about 25 pmol to 25 μmol. In addition, the subject kits may include an array of single-stranded probe nucleic acids (or a means for producing the same) wherein each probe has a RNA polymerase promoter domain and complement variable domain, as described above. Where the kit has a means for producing the template array, the kit may include a substrate having a planar surface, and one or more reagents necessary for synthesis of the probes, which may vary depending on the nature of the protocol to be used to generate the array. The kits may further include reagents necessary for producing labeled target nucleic acids, where such reagents may include reverse transcriptase, labeled dNTPs, etc. A set of instructions will also typically be included, where the instructions may be associated with a package insert and/or the packaging of the kit or the components thereof.

Although the foregoing invention has been described in some detail by way of illustration and example for purposes of clarity of understanding, it is readily apparent to those of ordinary skill in the art in light of the teachings of this invention that certain changes and modifications may be made thereto without departing from the scope of the appended claims.

What is claimed is:

1. A method for producing a plurality of ribonucleic acids, said method comprising:
(a) contacting:
(i) an array of at least two distinct features each comprising single-stranded nucleic acids immobilized on a surface of a solid support and having a surface proximal RNA polymerase promoter domain and a surface distal variable domain; with
(ii) nucleic acids complementary to said RNA polymerase promoter domain;
to produce a template array of overhang comprising duplex nucleic acids, wherein each overhang comprising duplex nucleic acid of said array comprises a double-stranded RNA polymerase promoter region and a single-stranded variable region overhang; and
(b) subjecting said template array to an in vitro transcription protocol to produce a product plurality of ribonucleic acids of differing sequence.

2. The method according to claim 1, wherein said single-stranded surface immobilized nucleic acids of said array further comprise a linking domain between said promoter and variable domains.

3. The method according to claim 1, wherein said single-stranded surface immobilized nucleic acids of said array further comprise a spacer between said surface proximal RNA polymerase promoter domain and said surface.

4. The method according to claim 1, wherein said immobilized nucleic acids of said features each have the same RNA polymerase promoter domain.

5. The method according to claim 1, wherein said RNA polymerase promoter domain is chosen from a T7, T3 and SP6 polymerase promoter domain.

6. The method according to claim 1, wherein said method further comprises subjecting said template array product of step (a) to primer extension reaction conditions prior to said subjecting step (b).

7. The method according to claim 1, wherein said method further comprises separating said product plurality from said template array.

8. The method according to claim 1, wherein product plurality comprises labeled ribonucleic acids.

9. The method according to claim 1, wherein said product plurality comprises unlabeled ribonucleic acids.

10. The method according to claim 1, wherein said single stranded surface immobilized nucleic acids of said features are described by the formula:
surface-Sₛ-R-L_{I}-V-5′
wherein:
S is a spacer domain;
s is an integer of 0 or 1;
R is said surface proximal RNA polymerase promoter domain;
L is a linking domain;
I is an integer of 0 or 1; and
V is said surface distal variable domain.

11. The method according to claim 1, wherein said array comprises features having a density of nucleic acid per feature ranging from about 10⁻³ to about 1 pmol/mm².

12. The method according to claim 1, where said method further comprises employing said product plurality in a differential gene expression analysis application.

13. The method according to claim 1, wherein said method further comprises employing said product plurality in a gene-silencing application.

14. An array comprising at least two distinct nucleic acid features each comprising single-stranded nucleic acids immobilized on a surface of substrate, wherein each of said surface immobilized single-stranded nucleic acids comprises a surface proximal RNA polymerase promoter domain and a surface distal variable domain.

15. The array according to claim 14, wherein said surface immobilized single-stranded nucleic acids are described by the formula:
surface-Sₛ-R-L_{I}-V-5′
wherein:
S is a spacer domain;
s is an integer of 0 or 1;
R is said surface proximal RNA polymerase promoter domain;
L is a linking domain;
I is an integer of 0 or 1; and
V is said surface distal variable domain;
wherein only said variable domain V of said surface immobilized single-stranded nucleic acids differs between features.

16. The array according to claim 15, wherein R is chosen from a T7, T3 and SP6 polymerase promoter domain.

17. The array according to claim 14, wherein said array comprises features having a density of nucleic acid per feature ranging from about 10⁻³ to about 1 pmol/mm².

18. A template array comprising at least two distinct nucleic acid features each comprising surface immobilized overhang comprising duplex nucleic acids, wherein each overhang comprising duplex nucleic acid of said array comprises a double-stranded RNA polymerase promoter region and a single-stranded variable region overhang.

19. The template array according to claim 14, wherein said surface immobilized overhang comprising duplex nucleic acids are described by the formula: 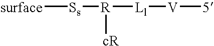
wherein:
S is a spacer domain;
s is an integer of 0 or 1;
R is said surface proximal RNA polymerase promoter domain;
cR is a nucleic acid complementary to said RNA polymerase promoter domain;
L is a linking domain;
I is an integer of 0 or 1; and
V is said surface distal variable domain;
wherein only said variable domain V of said surface immobilized single-stranded nucleic acids differs between features.

20. The array according to claim 19, wherein R is chosen from a T7, T3 and SP6 polymerase promoter domain.

21. The array according to claim 18, wherein said array comprises features having a density of nucleic acid per feature ranging from about 10⁻³ to about 1 pmol/mm².

22. A kit for use in producing a mixture of ribonucleic acids, said kit comprising:
(a) an array comprising at least two distinct nucleic acid features each comprising single-stranded nucleic acids immobilized on a surface of substrate, wherein each of said surface immobilized single-stranded nucleic acids comprises a surface proximal RNA polymerase promoter domain and a surface distal variable domain; and
(b) nucleic acids complementary to said RNA polymerase promoter domain.

23. The kit according to claim 22, wherein said kit further comprises a RNA polymerase.

24. The kit according to claim 22, wherein said kit further comprises ribonulcleotides.

25. The kit according to claim 24, wherein said ribonucleotides are labeled.

26. The kit according to claim 22, wherein said surface immobilized single-stranded nucleic acids are described by the formula:
surface-Sₛ-R-L_{I}-V-5′
wherein:
S is a spacer domain;
s is an integer of 0 or 1;
R is said surface proximal RNA polymerase promoter domain;
L is a linking domain;
I is an integer of 0 or 1; and
V is said surface distal variable domain;
wherein only said variable domain V of said surface immobilized single-stranded nucleic acids differs between features.

27. The kit according to claim 26, wherein R is chosen from a T7, T3 and SP6 polymerase promoter domain.

28. The kit according to claim 22, wherein said array comprises features having a density of nucleic acid per feature ranging from about 10⁻³ to about 1 pmol/mm².

29. A method of detecting the presence of a nucleic acid analyte in a sample, said method comprising:
(a) producing from said sample a target composition comprising:
(i) labeled deoxyribonucleic acid target molecules labeled with a first label; and
(ii) a ribonucleic acid reference labeled with a second label distinguishable from said first label, where said reference is produced according to the method of claim 1;
(b) contacting said target composition with a nucleic acid array;
(c) detecting any binding complexes on the surface of the said array to determine the presence of said nucleic acid analyte in said sample.

30. The method according to claim 29, wherein said method further comprises a data transmission step in which a result from a reading of the array is transmitted from a first location to a second location.

31. A method according to claim 30, wherein said second location is a remote location.

32. A method comprising receiving data representing a result of a reading obtained by the method of claim 29.


SEQUENCE LISTING (1 sequence)

SEQ ID NO: 1 - RNA, 17 nt, T7
  misc_feature 9: n = A,T,C or G
ugcaucaunaugaugca